(19) United States
(12) Patent Application Publication
(10) Pub. No.: US 2010/0145417 A1
(43) Pub. Date: Jun. 10, 2010

(54) LICE EXTERMINATION SYSTEM AND METHOD

(75) Inventors: Lori Kaufmann, Ra'anana (IL); Ram Oron, Nes Ziona (IL); Ariela Donval, Rosh Ha'ayin (IL); Doron Nevo, Ra'anana (IL); Moshe Oron, Rehovot (IL)

Correspondence Address: EDWARDS ANGELL PALMER & DODGE LLP, P.O. BOX 55874, BOSTON, MA, 02205, US

(73) Assignee: Y.K. HOLDINGS LTD., Ra'anana (IL)

(21) Appl. No.: 12/527,046

(22) PCT Filed: Feb. 17, 2008
(86) PCT No.: PCT/IL08/00206

§ 371 Date: Feb. 12, 2010

Related U.S. Application Data
(60) Provisional application No. 60/902,148, filed on Feb. 16, 2007.

(51) Int. Cl. A61N 5/06

(52) U.S. Cl. 607/91

(57) ABSTRACT

Apparatus is provided for treating an infested area (27) of a subject including a housing (20) and at least one heating element coupled to the housing (20). The hearing element is configured to emit light having an emission spectrum selected from the group consisting of: visible light and infrared light, which spectrum is better absorbed by an infestation selected from the group consisting of: lice, and lice eggs, and the tissue including at least one tissue selected from the group consisting of: skin of the subject, and hair of the subject. Other embodiment are also described.

### CROSS-REFERENCES TO RELATED APPLICATIONS

The present application claims priority from U.S. Provisional Patent Application 60/902,148 to Kaufmann, entitled, “Lice extermination system and method,” filed on Feb. 16, 2007, and is related to:

(a) U.S. Provisional Patent Application 60/709,353, entitled, “Lice extermination system and method,” filed Aug. 17, 2005,

(b) U.S. Provisional Patent Application 60/715,545, entitled, “Lice extermination system and method,” filed Sep. 8, 2005,

(c) U.S. Provisional Patent Application 60/771,937, entitled, “Lice extermination system and method,” filed Feb. 8, 2006, and

(d) PCT Patent Application PCT/IL2006/000964 to Kaufmann, entitled, “Lice extermination system and method,” filed Aug. 17, 2006.

These applications are all assigned to the assignee of the present application and are incorporated herein by reference.

### FIELD OF THE INVENTION

The present invention relates generally to treating insect infestations, and specifically to methods and apparatus for exterminating lice.

### BACKGROUND OF THE INVENTION

Human pediculosis is the infestation of the human scalp, body or pubic area with lice. Head lice lay eggs on the hair of the head. From the eggs hatch nymphs, which mature into adult lice. Lice infestations are conventionally treated with chemicals, such as shampoos, lotions, and creams containing various insecticides such as lindane, malathion, or permethrin, which generally kill the lice but not the eggs. Following treatment with these chemicals, the eggs are manually removed by careful examination of the affected area. In order for chemical treatment to be potentially effective, it generally must be repeated at least once, in order to exterminate lice that hatch from eggs that were overlooked during the manual egg removal. Lice are increasingly developing resistance to commonly used chemicals.

To overcome the difficulty of manually removing lice, eggs, and nits, numerous lice combs have been developed, including combs with finely spaced teeth, and combs with teeth having particular shapes (e.g., widening at the base, or diamond shaped). Some combs incorporate a magnifying glass to aid in finding eggs. Some combs incorporate an adhesive strip or spots that trap lice. Some combs incorporate a motor to coordinate vibration or meshing of teeth for ease of passage through hair, detangling of hair, and/or injuring lice. Some combs include a heating element for heating the teeth, in order to kill lice trapped between the teeth.

Some combs include a vacuum attachment for removing lice and fleas, guide lice to an adhesive strip in order to trap them, and/or guide lice through an insecticide-impregnated filter. Some combs include an electrocution device for remote electrocution of lice. Some combs include a rotating brush which brushes animal fleas onto an insecticide-impregnated sponge. Some combs have hollow teeth, through which the comb dispenses steam or hot air to the hair root, or an insecticide (see, for example, European Patent Application EP 1 002 477 A1 to Lorenzo, which is incorporated herein by reference). Some combs emit ultraviolet radiation designed to disinfect the head from pests and fungi.

Epilady® 2000, L.L.C. (Hatzor Haglilit, Israel) markets the Robi™ Comb Pro, an electronic lice comb that electrocutes lice. The company describes the comb as having two operation modes, one for lice detection and the other for lice extermination. Similarly, U.S. Pat. No. 5,318,051 to Koppel, which is incorporated herein by reference, describes the use of an electric current to remove lice and nits.

U.S. Pat. No. 6,053,180 to Kwan, which is incorporated herein by reference, describes a UV light-emitting source in or adjacent to the styling portion of a hair and/or animal fur care or styling appliance. The UV light-emitting source is described as capable of facilitating the killing of micro-organisms including spores, virus, fungus, bacteria and lice residing on hair and/or fur as well as on the appliance itself.

U.S. Pat. No. 5,353,817 to Kantor et al., which is incorporated herein by reference, describes a hand-held hair grooming device comprising a housing adapted to be held in the hand of the user, an array including a plurality of electrically conductive tines extending outwardly from the housing, apparatus for mounting the plurality of tines in the housing, apparatus for applying an electric potential between adjacent times whereby lice or other pests suitably disposed between adjacent tines are electrocuted, and apparatus for selectable displacement of some of the plurality of tines relative to others of said plurality of tines to assist in cleaning thereof.

U.S. Pat. Nos. 5,072,746 and 5,178,168 to Kantor, which are incorporated herein by reference, describe a hand-held hair grooming device comprising a housing adapted to be held in the hand of the user, an array of electrically conductive tines extending outwardly from the housing, apparatus for mounting the tines in the housing, and apparatus for applying an electric potential between adjacent tines whereby lice or other pests suitably disposed between adjacent tines are electrocuted.

U.S. Pat. No. 5,078,157 to Golan et al., which is incorporated herein by reference, describes apparatus for treating hair including a handle, hair separation apparatus associated with the handle and extending longitudinally along an axis, apparatus for oscillating the hair separation apparatus about the axis so as to bring the hair separation apparatus into repeated engagement with a group of hairs so as to ease passage of the separation apparatus therethrough, and apparatus for preventing the hair separation apparatus from striking a portion of skin to which the hairs are attached. The hair separation apparatus is oscillated about the axis at a relatively high frequency, of, typically, greater than 4,000 cycles per minute and preferably greater than 10,000 cycles per minute, and at a relatively small amplitude, of, typically, no larger than about 5 degrees and, preferably, no larger than 2 degrees.

U.S. Pat. No. 5,628,332 to Debourg et al., which is incorporated herein by reference, describes techniques for destroying parasites present in a mass of hair or fur growing from a skin surface, the device comprising a handling body; a comb fixed to the body and comprising of a plurality of teeth, the teeth having portions adapted to be displaced through the mass of hair or fur when the device is in use; and a source of energy for at least locally raising the temperature of the hair or fur when the teeth are displaced through the mass of hair or fur, wherein at least part of the portions of the teeth are thermally conductive and are operatively associated with a source of energy for supplying sufficient heat energy to destroy parasites on the hair or fur which is in contact with the portions.

U.S. Pat. No. 6,685,969 and US Patent Application Publication 2004/0126403 to Van Scoik et al., which are incorporated herein by reference, describe the removal of ectoparasites, such as fleas, ticks, mites and lice, from a human or animal host by applying a composition that causes the temperature of a treated area to become higher or lower than normal. In one embodiment, treatment is effected by applying a composition that releases heat when contacted with water. The elevated or decreased temperature immobilizes the parasites and facilitates their mechanical removal, such as by combing.

U.S. Pat. No. 6,689,394 to Van Scoik et al., which is incorporated herein by reference, describes the removal of ectoparasites, such as fleas, ticks, mites and lice, from a human or animal host by applying a composition to the affected area that causes desiccation of the organism. In one embodiment, treatment is effected by applying a substantially anhydrous composition that has a strong affinity for water. As the composition hydrates, it desiccates the pests and facilitates their mechanical removal, such as by combing.

U.S. Pat. Nos. 6,875,421, 6,440,388, and 5,997,846 to Burns et al., which are incorporated herein by reference, describe a method for detecting chitin-containing organisms on an area of a person or animal by contacting the particular area with a dye that is capable of binding to chitin and emitting fluorescence upon exposure to light. If a chitin-containing organism is present in the treated area, the chitin of the organism binds the dye and, upon exposure to light, the chitin-containing organisms may be visualized and removed.

U.S. Pat. Nos. 6,541,455, 6,350,734, 6,303,581, and 6,265,384 to Pearlman, which are incorporated herein by reference, describe methods and kits for removing, treating, or preventing head lice infestations, including topically applying to the lice-infested area an effective amount of a dryable pediculostatic agent for a time sufficient to immobilize the lice, drying the agent onto the application site, and removing the dried agent, thereby removing the lice and nits.

U.S. Pat. No. 5,261,427 to Dolev, which is incorporated herein by reference, describes a lice comb device constructed as a portable, hand-held housing containing a blower heater, to heat and direct a stream of heated air toward a set of comb teeth attached to the housing. Each of the comb teeth is formed with a hollow cavity having an opening at its end. The heated air stream is directed via the comb tooth and exits at the opening, where it is deflected away from the scalp by a concave surface, so that it impinges on lice and lice eggs attached near the hair root. The temperature of the heated air destroys the lice and lice eggs.

U.S. Pat. No. 5,972,987 to Reid et al., which is incorporated herein by reference, describes a method for removing lice eggs from the hair of an infested human or animal using a nit-visualizing composition. The composition involves the use of certain dyes which have an affinity to the surface of nits, thereby enabling a second individual to more easily see and remove the eggs during a combing or other removal process. A colored material is dispersed within a water- or alcohol-based solvent and, in one embodiment, a liquefied propellant as well. The composition is applied to the hair of the infested human or animal, and then removed after drying by a process of brushing or washing. The colored material which adsorbs to the chitinous exoskeleton and binding cement of the lice eggs remains on the nits, thereby facilitating visual identification and removal of the lice eggs from hair.

U.S. Pat. No. 5,997,847 to Spiesel, which is incorporated herein by reference, describes a method for detecting the infestation of a host by arthropod ectoparasites using a fluorescent dye which stains the ectoparasites and/or their eggs but not the adjacent skin or hair to which they are attached. The dye can be incorporated into a shampoo or a rinsing solution and is applied to the scalp or other region of the host. After a suitable period of time has elapsed, the dye-containing solution or shampoo is rinsed off and the hair and adjacent area of the host examined under ultraviolet or near-ultraviolet light. Such illumination will cause the stained ectoparasites and/or eggs to glow and, thus, become easily detectable for diagnosis and subsequent removal. The method may be applied to detect scabies (i.e. skin-burrowing mites), head lice, body and pubic lice and to any other arthropod ectoparasites and their eggs containing a substantial percentage of chitin.

### SUMMARY OF THE INVENTION

In embodiments of the present invention, a lice and eggs extermination device comprises a housing, an optical guide, and a light source having an emission spectrum that is better absorbed by living lice and/or eggs than by human or animal skin and hair. Typically, the emission spectrum is selected for relatively high absorption by chitin, a polysaccharide present in the exoskeleton of lice and eggs, but not in human or animal hair or skin. The optical guide is typically adapted to distribute the light from a plurality of directions, in order to provide uniform and thorough exposure of an infested area, even if the area is completely or partially shielded by hair. The emission spectrum typically includes a wavelength range within the visible or infrared (near, medium, or far) spectra. The extermination device is typically either hand-held or stand-alone. For some applications, the extermination device is incorporated into a comb, a helmet, gloves, or any other lice and eggs removal device.

For some applications in which the housing of the extermination device comprises a helmet, the housing comprises a plurality of hair separators, e.g., ridges, tines or teeth, which separate portions of the subject's hair. The plurality of hair separators are typically aligned adjacent to each other and longitudinally with respect to the extermination device, and permit hair to be introduced therebetween when the helmet is advanced, for example, in a dorsal to ventral direction with respect to the head of the subject. Once advanced through the hair of the subject, the helmet typically remains immobile during the treatment procedure.

Alternatively, the extermination device comprises a hand-held housing, e.g., a comb or a glove, which comprises a plurality of hair separators, e.g., tines or teeth, configured to separate and treat hair as the comb is advanced through the hair of the subject. During the treatment procedure, the hand-held housing is configured to be advanced along the head of the subject. Alternatively, the hand-held housing remains fixed to a particular location on the head of the subject during the treatment procedure, and is then advanced to another location.

For some applications, a thermally-conductive material coats the extermination device. Alternatively, the thermally-conductive material is incorporated within the material of the housing of the extermination device.

For some applications, at least a portion of the plurality of hair separators is coupled to the thermally-conductive material, which is operatively associated with a source of energy, either remotely or directly coupled thereto. Alternatively, the thermally-conductive material is not coupled to the hair separators and is applied to the hair of the subject in conjunction with placing the housing around a portion of the head of the subject. Sufficient heat energy is supplied such that it effects an emission, from the portion of the hair separators, of infrared waves having wavelengths of about 9 to 10 microns, which are configured to destroy parasites on hair in contact with or in line of sight of the portions. Alternatively or additionally, the extermination device is configured to destroy parasites on hair that are not in contact with or in line of sight of the extermination device.

Typically, the plurality of hair separators provide increased surface area for dissipation of heat and consequently, propagation of infrared waves evenly and relatively quickly through a mass of hair and toward the infested area.

There is therefore provided, in accordance with an embodiment of the present invention, apparatus for treating an infested area of a subject, including:

a head cover; and

a plurality of tines coupled to the head cover and including respective heating elements that are configured to emit infrared light having a wavelength less than 10 microns.

There is further provided, in accordance with an embodiment of the present invention, apparatus for treating an infested area of a subject, including:

a housing; and

at least one heating element coupled to the housing and configured to emit light having an emission spectrum selected from the group consisting of: visible light and infrared light, which spectrum is better absorbed by an infestation of the area than by tissue of the subject, the infestation including at least one infestation selected from the group consisting of: lice, and lice eggs, and the tissue including at least one tissue selected from the group consisting of: skin of the subject, and hair of the subject.

In an embodiment, the infestation includes both lice and eggs, and the heating element is configured to emit the light having the emission spectrum that is better absorbed by the infestation than by the tissue.

In an embodiment, the apparatus further includes a cooling unit, which is configured to remove excess heat generated by the heating element.

In an embodiment, the housing includes a glove.

In an embodiment, the housing includes a head cover.

In an embodiment, the housing includes a plurality of hair separators configured to displace the hair of the subject.

In an embodiment, the at least one heating element includes a plurality of heating elements, and at least a portion of the plurality of hair separators include the plurality of heating elements.

In an embodiment, the plurality of hair separators are configured to facilitate application of the emitted light to the infestation by moving the hair of the subject and separating the hair into portions, and the heating element emits the light to the portions.

In an embodiment, the plurality of hair separators are configured to raise the hair of the subject.

In an embodiment, the plurality of hair separators includes a comb.

In an embodiment, the plurality of hair separators includes a single-port suction applicator.

In an embodiment, the plurality of hair separators includes a multi-port suction applicator.

In an embodiment, the plurality of hair separators includes a flexible fastener.

In an embodiment, the plurality of hair separators includes one or more hair clips.

In an embodiment, the plurality of hair separators are spaced apart by a distance of more than 1 mm.

In an embodiment, the apparatus further includes a thermally-conductive material, which is configured to conduct heat from the at least one heating element to the housing.

In an embodiment, the thermally-conductive material coats the housing.

In an embodiment, the thermally-conductive material is integrated within the housing.

In an embodiment, at least a portion of the plurality of hair separators includes the thermally-conductive material.

In an embodiment, the apparatus further includes a comb coupled to the heating element.

In an embodiment, the at least one heating element is configured to be integrated into the comb.

In an embodiment, the comb is configured to be coupled to the housing.

In an embodiment, the comb includes tines, and the heating element is configured to heat the tines.

In an embodiment, the at least one heating element is configured to raise its temperature to below 70 C.

In an embodiment, the at least one heating element is configured to raise its temperature to below 60 C.

In an embodiment, in response to the raised temperature, the heating element emits light having an emission spectrum that has a peak between 9 microns and 10 microns.

In an embodiment, the at least one heating element is configured to raise its temperature to between 40 C and 70 C.

In an embodiment, the at least one heating element is configured to raise its temperature to between 40 C and 55 C.

In an embodiment, the at least one heating element is configured to raise its temperature to between 55 C and 70 C.

There is still further provided, in accordance with an embodiment of the present invention apparatus for treating an infested area of a subject, the apparatus including:

a housing; and

at least one heating element coupled to the housing and configured to emit light having an emission spectrum selected from the group consisting of: visible light and infrared light, which spectrum is better absorbed by chitin than by tissue of the subject.

There is yet further provided, in accordance with an embodiment of the present invention, a method for treating a subject, the method including:

identifying an infestation of an area of the subject, the infestation including at least one infestation selected from the group consisting of: lice, and lice eggs; and

emitting light in a vicinity of the infestation by generating heat in the vicinity of the infestation, the light having an emission spectrum selected from the group consisting of: visible light and infrared light, which spectrum is better absorbed by the infestation of the area than by tissue of the subject, the tissue including at least one tissue selected from the group consisting of: skin of the subject, and hair of the subject.

In an embodiment, generating the heat includes, responsively to the generating, emitting light having an emission spectrum that has a peak between 9 microns and 10 microns.

In an embodiment, emitting the light includes emitting the light using at least one lighting element selected from the group consisting of: an incandescent lighting element, a laser, and an LED.

In an embodiment, generating heat in the vicinity of the infestation includes generating the heat using at least one heating element.

In an embodiment, the method includes facilitating application of the emitted light to the infestation by moving the hair of the subject.

In an embodiment, moving the hair includes raising the hair of the subject.

In an embodiment, emitting the light includes emitting the light from a comb, and the method includes combing the area with the comb while the light is being emitted.

In an embodiment, emitting the light includes emitting the light from tines of the comb.

In an embodiment, the emission spectrum has a wavelength between 0.5 and 10 microns, and emitting the light includes emitting the light having the emission spectrum having a wavelength between 0.5 and 10 microns.

In an embodiment, the emission spectrum has a wavelength between 6 and 7 microns, and emitting the light includes emitting the light having the emission spectrum having a wavelength between 6 and 7 microns.

In an embodiment, the emission spectrum has a wavelength between 3 and 3.5 microns, and emitting the light includes emitting the light having the emission spectrum having a wavelength between 3 and 3.5 microns.

In an embodiment, the emission spectrum has a wavelength between 3 and 3.5 microns and between 6 and 7 microns, and emitting the light includes emitting the light having the emission spectrum having a wavelength between 3 and 3.5 microns and between 6 and 7 microns.

In an embodiment, the emission spectrum has a wavelength between 9 and 10 microns, and emitting the light includes emitting the light having the emission spectrum having a wavelength between 9 and 10 microns.

There is additionally provided, in accordance with an embodiment of the present invention, a method for treating a subject, the method including:

identifying an infestation of an area of the subject; and

emitting light in a vicinity of the infestation by generating heat in the vicinity of the infestation, the light having an emission spectrum selected from the group consisting of: visible light and infrared light, which spectrum is better absorbed by chitin than by tissue of the subject, the tissue including at least one tissue selected from the group consisting of skin of the subject, and hair of the subject.

There is also provided, in accordance with an embodiment of the present invention, apparatus for treating an infested area of a subject, the apparatus including:

a housing;

an illumination unit, coupled to the housing, and including a light source configured to emit light having an emission spectrum selected from the group consisting of: visible light and infrared light, which spectrum is better absorbed by an infestation of the area than by tissue of the subject, the infestation including at least one infestation selected from the group consisting of lice, and lice eggs, and the tissue including at least one tissue selected from the group consisting of: skin of the subject, and hair of the subject; and

an optical guide unit, coupled to the housing, and configured to distribute the light to the area.

In an embodiment, the infestation includes both lice and eggs, and the light source is configured to emit the light having the emission spectrum that is better absorbed by the infestation than by the tissue.

For some applications, the apparatus includes a cooling unit, which is configured to remove excess heat generated by the light source. For some applications, the apparatus includes a comb, a head cover, and/or a glove, with which the optical guide unit is integrated. For example, the comb may include tines, and the optical guide unit may include light guides that are integrated into the tines. For some applications, the light source includes at least one lighting element selected from the group consisting of: an incandescent lighting element, a laser, and an LED. For some applications, the optical guide unit is configured to distribute the light from a plurality of directions.

In an embodiment, the emission spectrum has a wavelength between 0.5 and 10 microns, and the light source is configured to emit the light having the emission spectrum having a wavelength between 0.5 and 10 microns. For some applications, the emission spectrum has a wavelength between 6 and 7 microns, and the light source is configured to emit the light having the emission spectrum having a wavelength between 6 and 7 microns. Alternatively, for some applications, the emission spectrum has a wavelength between 3 and 3.5 microns, and the light source is configured to emit the light having the emission spectrum having a wavelength between 3 and 3.5 microns. Further alternatively, for some applications, the emission spectrum has a wavelength between 9 and 10 microns, and the light source is configured to emit the light having the emission spectrum having a wavelength between 9 and 10 microns. Still further alternatively, for some applications, the emission spectrum has a wavelength between 3 and 3.5 microns and between 6 and 7 microns, and the light source is configured to emit the light having the emission spectrum having a wavelength between 3 and 3.5 microns and between 6 and 7 microns.

There is also provided, in accordance with an embodiment of the present invention, apparatus for treating an infested area of a subject, the apparatus including:

a housing;

an illumination unit, coupled to the housing, and including a light source configured to emit light having an emission spectrum selected from the group consisting of: visible light and infrared light, which spectrum is better absorbed by chitin than by tissue of the subject; and

an optical guide unit, coupled to the housing, and configured to distribute the light to the area.

For some applications, the apparatus includes a cooling unit, which is configured to remove excess heat generated by the light source. For some applications, the apparatus includes a head cover, with which the optical guide unit is integrated. Alternatively; the apparatus includes a glove, with which the optical guide unit is integrated. Further alternatively, the apparatus includes a comb, with which the optical guide unit is integrated. For example, the comb may include tines, and the optical guide unit may include light guides that are integrated into the tines.

For some applications, the light source includes at least one lighting element selected from the group consisting of an incandescent lighting element, a laser, and an LED. For some applications, the optical guide unit is configured to distribute the light from a plurality of directions.

In an embodiment, the emission spectrum has a wavelength between 0.5 and 10 microns, and the light source is configured to emit the light having the emission spectrum having a wavelength between 0.5 and 10 microns. For some applications, the emission spectrum has a wavelength between 6 and 7 microns, and the light source is configured to emit the light having the emission spectrum having a wavelength between 6 and 7 microns. Alternatively, the emission spectrum has a wavelength between 3 and 3.5 microns, and the light source is configured to emit the light having the emission spectrum having a wavelength between 3 and 3.5 microns. Further alternatively, the emission spectrum has a wavelength between 3 and 3.5 microns and between 6 and 7 microns, and the light source is configured to emit the light having the emission spectrum having a wavelength between 3 and 3.5 microns and between 6 and 7 microns. Still further alternatively, the emission spectrum has a wavelength between 9 and 10 microns, and the light source is configured to emit the light having the emission spectrum having a wavelength between 9 and 10 microns.

In an embodiment, the apparatus includes a component configured to facilitate application of the emitted light to the infestation by moving the hair of the subject. For example, the component may be configured to raise and/or separate the hair of the subject. For some applications, the component includes a comb, a single-port suction applicator, a multi-port suction applicator, a flexible fastener, and/or one or more hair clips.

There is further provided, in accordance with an embodiment of the present invention, a method for treating a subject, the method including:

identifying an infestation of an area of the subject, the infestation including at least one infestation selected from the group consisting of: lice, and lice eggs;

emitting light having an emission spectrum selected from the group consisting of: visible light and infrared light, which spectrum is better absorbed by the infestation of the area than by tissue of the subject, the tissue including at least one tissue selected from the group consisting of: skin of the subject, and hair of the subject; and

distributing the light to the area.

There is additionally provided, in accordance with an embodiment of the present invention, a method for treating a subject, the method including:

identifying an infestation of an area of the subject;

emitting light having an emission spectrum selected from the group consisting of: visible light and infrared light, which spectrum is better absorbed by chitin than by tissue of the subject; and

distributing the light to the area.

In an embodiment, distributing the light includes facilitating application of the emitted light to the infestation by moving the hair of the subject, e.g., raising and/or separating the hair. For some applications, moving the hair includes moving the hair using a comb, applying suction to the hair using a single- or multi-port suction applicator, transiently binding the hair to a flexible fastener, and/or moving the hair using one or more hair clips.

There is still additionally provided, in accordance with an embodiment of the present invention, apparatus for treating an infested area of a subject, including:

a housing; and

at least one heating element coupled to the housing and configured to emit light having an emission spectrum selected from the group consisting of: visible light and infrared light, which spectrum is better absorbed by an infestation of the area than by tissue of the subject, the infestation including at least one infestation selected from the group consisting of: lice, and lice eggs, and the tissue including at least one tissue selected from the group consisting of: skin of the subject, and hair of the subject.

In an embodiment, the infestation includes both lice and eggs, and the heating element is configured to emit the light having the emission spectrum that is better absorbed by the infestation than by the tissue.

In an embodiment, the apparatus includes a cooling unit, which is configured to remove excess heat generated by the heating element.

In an embodiment, the housing includes a glove.

In an embodiment, the housing includes a head cover.

In an embodiment, the housing includes a plurality of hair separators configured to displace the hair of the subject.

In an embodiment, the at least one heating element includes a plurality of heating elements, and at least a portion of the plurality of hair separators include the plurality of heating elements.

In an embodiment, the plurality of hair separators are configured to facilitate application of the emitted light to the infestation by moving the hair of the subject and separating the hair into portions, and the heating element emits the light to the portions.

In an embodiment, the plurality of hair separators are configured to raise the hair of the subject.

In an embodiment, the plurality of hair separators includes a comb.

In an embodiment, the plurality of hair separators includes a single-port suction applicator.

In an embodiment, the plurality of hair separators includes a multi-port suction applicator.

In an embodiment, the plurality of hair separators includes a flexible fastener.

In an embodiment, the plurality of hair separators includes one or more hair clips.

In an embodiment, the plurality of hair separators are spaced apart by a distance of more than 1 mm.

In an embodiment, the apparatus includes a thermally-conductive material, which is configured to conduct heat from the at least one heating element to the housing.

In an embodiment, the thermally-conductive material coats the housing.

In an embodiment, the thermally-conductive material is integrated within the housing.

In an embodiment, at least a portion of the plurality of hair separators includes the thermally-conductive material.

In an embodiment, the apparatus includes a comb coupled to the heating element.

In an embodiment, the at least one heating element is configured to be integrated into the comb.

In an embodiment, the comb is configured to be coupled to the housing.

In an embodiment, the comb includes tines, and the heating element is configured to heat the tines.

In an embodiment, the at least one heating element is configured to raise its temperature to below 60 C.

In an embodiment, in response to the raised temperature, the heating element emits light having an emission spectrum that has a peak between 9 microns and 10 microns.

In an embodiment, the at least one heating element is configured to raise its temperature to between 40 C and 55 C.

In an embodiment, the at least one heating element is configured to raise its temperature to between 40 C and 50 C.

There is yet additionally provided, in accordance with an embodiment of the present invention, apparatus for treating an infested area of a subject, the apparatus including:

a housing; and

at least one heating element coupled to the housing and configured to emit light having an emission spectrum selected from the group consisting of visible light and infrared light, which spectrum is better absorbed by chitin than by tissue of the subject.

The present invention will be more fully understood from the following detailed description of embodiments thereof, taken together with the drawings, in which:

### BRIEF DESCRIPTION OF THE DRAWINGS

FIG. 1 is a schematic block diagram of a lice and eggs extermination system, in accordance with an embodiment of the present invention;

FIG. 2 is a graph showing a transmittance spectrum of chitin, for use in accordance with an embodiment of the present invention;

FIGS. 3A and 3B are schematic cross-sectional illustrations of a light source of the system of FIG. 1, in accordance with respective embodiments of the present invention;

FIGS. 4A and 4B are schematic cross-sectional illustrations of an illumination unit of the system of FIG. 1, in accordance with respective embodiments of the present invention;

FIG. 5 is a schematic illustration of a configuration of an optical guide unit of the system of FIG. 1, in accordance with an embodiment of the present invention; and

FIGS. 6 and 7A-C are schematic illustrations of the system of FIG. 1, comprising a head cover, in accordance with respective embodiments of the present invention.

### DETAILED DESCRIPTION OF EMBODIMENTS

FIG. 1 is a schematic block diagram of a lice and eggs extermination system 10, in accordance with an embodiment of the present invention. System 10 comprises a housing 20, an illumination unit 22, an optical guide unit 24, and a power supply 26. Optical guide unit 24 distributes light, symbolically represented by rays 44, to an infested area 27, such as a head of a subject.

For some applications, housing 20 is remotely or directly coupled to a heating element (not shown). Typically, housing 20 comprises a lightweight material (e.g., foam, plastic, cloth), and a thermally-conductive material. For some applications, the thermally-conductive material coats the lightweight material of housing 20. Alternatively, the thermally-conductive material is integrated within the lightweight material of housing 20. When housing 20 is heated, infrared light is emitted by the housing and radiated toward infested area 27. For example, infrared light having wavelengths between about 9 microns and about 10 microns is typically generated secondary to a temperature range of between 40 C and 70 C, e.g., between 40 C and 55 C or between 55 C and 70 C.

Illumination unit 22 comprises at least one light source 28, which typically comprises at least one incandescent light source, at least one laser, and/or at least one LED. For some applications, such as in which light source 28 comprises an incandescent light source, illumination unit 22 further comprises at least one optical filter 30, which allows passage of only one or more desired spectral bands. Filter 30 is typically not necessary for applications in which light source 28 comprises a laser, which emits light of a selected wavelength. Illumination unit 22 provides the light either in pulses or substantially continuously. For some applications, system 10 comprises a cooling unit 32, such as a blower or water cooling system, to remove excess heat generated by light source 28.

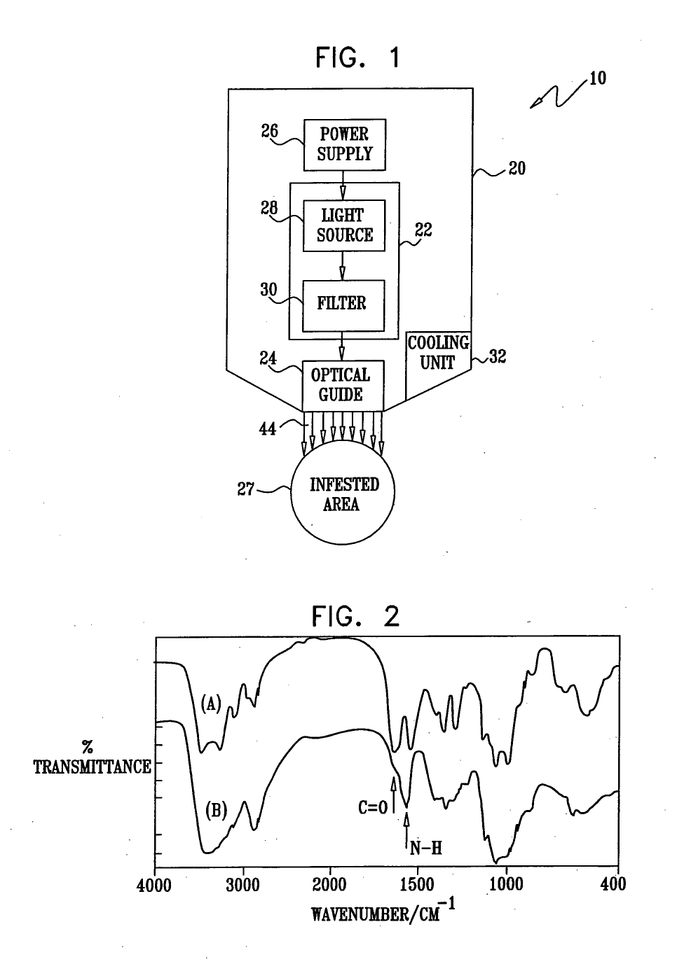

Reference is made to FIG. 2, which is a graph showing a transmittance spectrum of chitin, measured in accordance with an embodiment of the present invention. Illumination unit 22 is configured to emit light having an emission spectrum that is better absorbed by lice and/or eggs than by human or animal tissue, such as skin and hair. Typically, the emission spectrum is selected for relatively high absorption by chitin, a polysaccharide present in the exoskeleton of lice and eggs, but not in human or animal hair or skin. Therefore, the emission spectrum is typically better absorbed by the infestation than by tissue of the subject. Typically, the illumination is set to a level that is sufficient to exterminate lice and/or eggs, but insufficient to cause pain or tissue damage to the subject.

The following table highlights several of the peaks in the transmittance spectrum of the graph of FIG. 2:

TABLE 1

| | Wavenumber | Wavelength | |
|---|---|---|---|
| Peak # | [cm\hat{ }−1] | [microns] | % Transmittance |
| 1 | 3305.9 | 3.026 | 13.7 |
| 2 | 2963.1 | 3.375 | 14.3 |
| 3 | 2929.6 | 3.414 | 14.6 |
| 4 | 1650.3 | 6.06 | 10.0 |
| 5 | 1538.5 | 6.5 | 11.6 |
| 6 | 1396.4 | 7.161 | 15.0 |
| 7 | 1000.0 | 10.0 | 23.0 |

The emission spectrum of light emitted by system 10 is either discrete or continuous, and typically includes a wavelength range within the visible or infrared (near, medium, or far) spectra. For example, the wavelength range may fall between about 0.5 and about 10 microns, such as between about 3 and about 3.5 microns, between about 6 and about 7 microns, and/or between about 9 and about 10 microns.

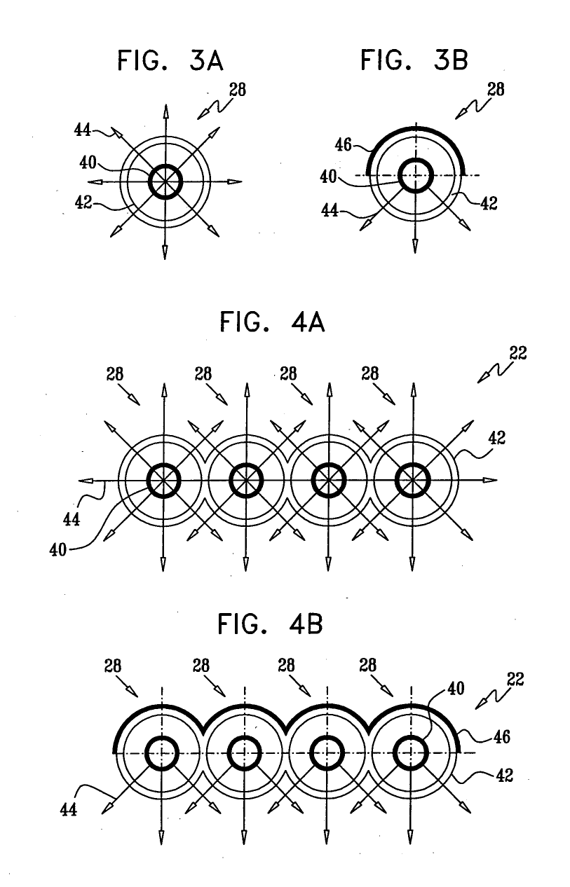

FIGS. 3A and 3B are schematic cross-sectional illustrations of light source 28, in accordance with respective embodiments of the present invention. In these embodiments, light source 28 comprises an incandescent light source, which is heated to a temperature appropriate for emitting the selected emission spectrum. The incandescent light source comprises a filament 40, which comprises, for example, tungsten, tantalum, or molybdenum, and which is coiled (e.g., as a spiral) and heated by an electrical current. Filament 40 is surrounded by a tube 42, which comprises infrared-transparent material, such as quartz, silicon, gallium arsenide (GaAs), germanium, or sapphire. For example, light source 28 may, comprise Medium Wave twin-tube emitter or Short Wave emitter “Quatro” No. 45131608, both manufactured by Heraeus Holding GmbH (Hanau, Germany), and system 10 may comprise cooling unit 32, such as a blower or water cooling system. In the unshielded configuration shown in FIG. 3A, light source 28 transmits light, symbolically represented by rays 44, in all directions. In the configuration shown in FIG. 3B, light source 28 further comprises a coating 46 that covers a portion of tube 42, such that light is transmitted in only a portion of directions, in a half-circle. Coating 46 is typically reflective, and comprises, for example, gold, aluminum, or silver.

FIGS. 4A and 4B are schematic cross-sectional illustrations of illumination unit 22, in accordance with respective embodiments of the present invention. In the embodiment shown in FIG. 4A, illumination unit 22 comprises a plurality of unshielded light sources 28, such as described hereinabove with reference to FIG. 3A. In the embodiment shown in FIG. 4B, illumination unit 22 comprises a plurality of shielded light sources 28, such as described hereinabove with reference to FIG. 3B. The configurations shown in FIGS. 4A and 4B are typically used when a single light source 28 is insufficient to supply the required energy.

Reference is again made to FIG. 1. Light emitted from illumination unit 22 passes through optical guide unit 24, which is typically adapted to distribute incoming light from a plurality of directions, in order to provide uniform (i.e., uniform energy density (watts/cm2)) and thorough exposure of infested area 27, even when the area is shielded by hair Optical guide unit 24 comprises either image-forming optics or non-image-forming optics.

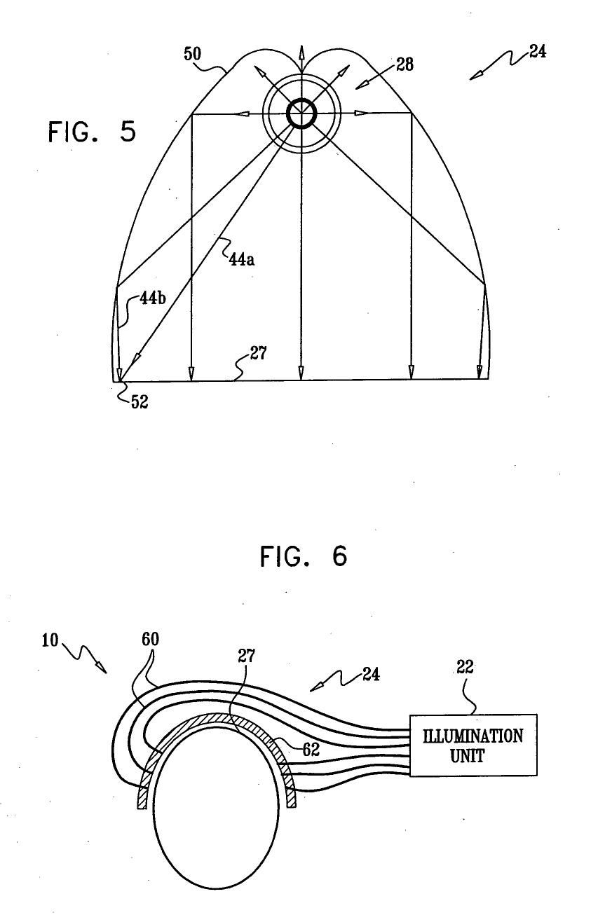

Reference is made to FIG. 5, which is a schematic illustration of a configuration of optical guide unit 24, in accordance with an embodiment of the present invention. In this embodiment, optical guide unit 24 comprises a non-imaging optical illuminator 50, configured to distribute light of lamp 28 substantially uniformly on infested area 27. For example, light rays 44a and 44b reach a point 52 from different directions. Because the light reaches substantially all points of infested area 27 from a plurality of directions, the light typically reaches areas that may be shielded in one or more directions by an obstacle, such as hair.

Reference is made to FIG. 6, which is a schematic illustration of an embodiment of system 10 in which optical guide unit 24 comprises one or more optical fibers 60. In this embodiment, system 10 comprises a head cover 62, adapted to surround at least a portion of infested area 27, which is typically a head of the subject. Head cover 62 is typically shaped like a helmet.

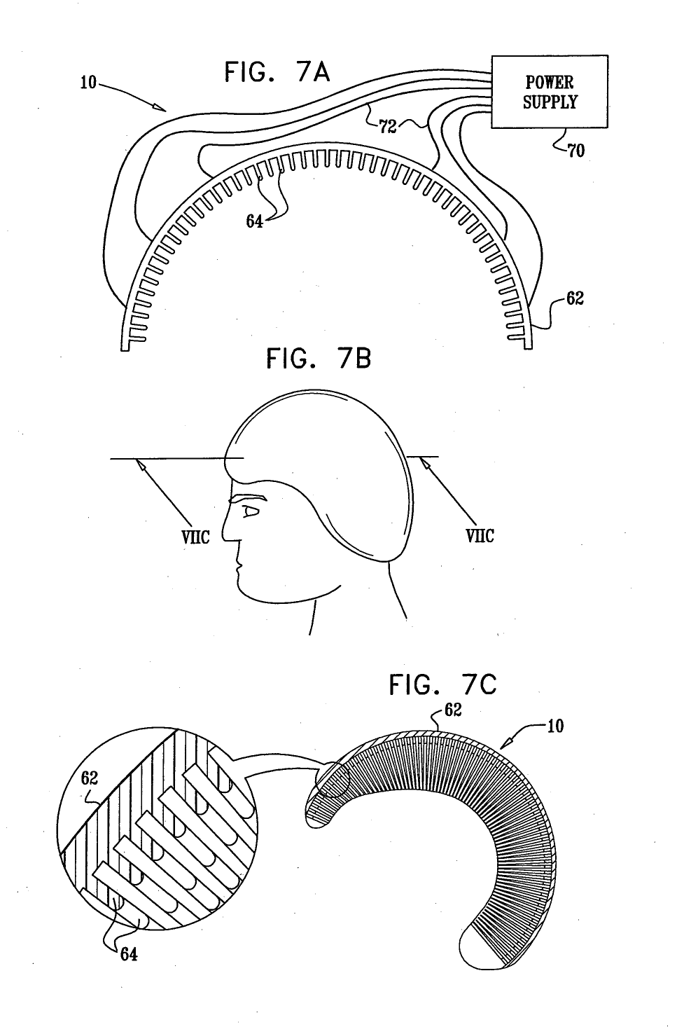

Reference is now made to FIGS. 7A-7C, which are schematic illustrations of an embodiment of system 10 in which head cover 62 comprises a plurality of hair separators 64 (e.g., teeth, tines, or ridges).

FIGS. 7B and 7C show head cover 62 comprising a helmet and the plurality of hair separators 64. Typically, hair separators 64 are successively disposed longitudinally with respect to head cover 62 upon an inner surface thereof. For some applications, hair separators 64 comprise an array of teeth or tines which are disposed along portions of, e.g., the entire, inner surface of the helmet.

Reference is again made to FIG. 7A-7C. The plurality of hair separators 64 function mechanically similarly to teeth of a comb, in that hair separators 64 permit hair to be introduced therebetween as head cover 62 is advanced, for example, in a dorsal to ventral direction with respect to the head of the subject. Once positioned on the head of the subject, head cover 62 remains immobile during the treatment procedure. For applications in which head cover 62 comprises a hand-held housing (e.g., a glove or a comb), head cover 62 is configured to be advanced around the head of the subject during the treatment procedure.

Typically, head cover 62 comprises a lightweight material (e.g., plastic, foam, or cloth), and is coated with a thermally-conductive material enabling hair separators 64 to be heated by at least one heating element (not shown). Alternatively, the thermally-conductive material is incorporated within the lightweight material of head cover 62.

As shown in FIG. 7A, system 20 comprises a power supply 70 which supplies energy to the heating element. For some applications, the at least one heating element comprises a plurality of heating elements which are directly coupled to at least a portion of the plurality of hair separators 64 by being incorporated into head cover 62. For some embodiments of this particular application, power supply 70 is disposed within head cover 62 and supplies energy to the heating elements. Alternatively, power supply 70 is disposed at a remote location with respect to head cover 62 and supplies energy to the heating elements via one or more electrical leads 72.

Typically, the heating element heats the housing by heating the at least a portion of hair separators 64 to a desired temperature range below 60 C. Typically the temperature range is between 40 C and 70 C, e.g., between 40 C and 55 C or between 55 C and 70 C. Secondary to the heating, infrared light is emitted from the housing having wavelengths corresponding to the range of temperature applied to hair separators 64. Typically, a range of between 40 C and 70 C, e.g., between 40 C and 55 C, emits infrared light having wavelengths between about 9 microns and about 10 microns. Infrared radiation emitted in this manner is differentially absorbed by the chitin of the lice and the lice eggs.

Typically, the plurality of hair separators 64 facilitate application of the emitted light to the infested area by moving the hair of the subject and separating the hair into portions. Thus for some applications, the emitted light is applied to portions of the hair directly in contact with hair separators 64. Alternatively or additionally, the emitted light is applied to potions of the hair which are in line of sight of, but not in contact with, hair separators 64. For some applications, the plurality of hair separators 64 are configured to raise the hair of the subject.

For some applications, the plurality of hair separators 64 comprises a comb.

In some embodiments, the plurality of hair separators 64 comprises a suction applicator, e.g., single-port or a multi-port suction applicator.

For some applications, the plurality of hair separators 64 comprises a flexible hair fastener.

In some embodiments, the plurality of hair separators 64 comprises one or more hair clips.

Typically, hair separators 64 are spaced apart by a distance of more than 1 mm, e.g., 3 mm. A large number of hair separators 64 generally function to provide increased surface area for enhancing the rate of radiating the infrared light to the lice and the lice eggs.

The following table shows results of a lice elimination experiment, performed in accordance with an embodiment of the present invention:

TABLE 2

| | | Elimination time |
|---|---|---|
| | | (Experimental) |
| Temperature [C.] | Wavelength [um] | [min] |
| 53.5 | 8.88 | 0.8 |
| 52 | 8.92 | 1.0 |
| 50 | 8.97 | 1.3 |
| 46 | 9.08 | 2.3 |

As shown in Table 2, the experimental elimination time is quite low. The low experimental elimination time is attributed by the inventors to the radiation effect of the emitted spectrum having the wavelength range of between about 9 microns and 10 microns corresponding to the applied temperature range. Additionally, the low experimental elimination time is attributed by the inventors to the radiation effect in combination with the increased surface area provided by hair separators 64 of head cover 62.

In an embodiment of the present invention, system 10 is configured to supply light having a flux of between about 0.08 and about 0.24 watts/cm2 over the 1 to 7 micron wavelength region, with a peak value at about 2.5 micron (medium wave emitter). In an embodiment, system 10 is configured to supply light having a flux of between about 0.23 and about 0.46 watts/cm2 over the 0.5 to 5 micron wavelength region, with a peak value at about 1.2 microns (short wave emitter).

The following table shows results of a lice mortality experiment, performed in accordance with an embodiment of the present invention:

TABLE 3

| Experi- | | | Light | | | |
|---|---|---|---|---|---|---|
| mental | | Filter | flux | Time | Mortality [%] | |
| Group | Lamp | [On/Off] | [W/cm2] | [min] | Lice | Eggs |
| 1 | Short IR | Off | 0.45 | 2 | 100 | 100 |
| 2 | Medium IR | Off | 0.11 | 3 | 100 | 100 |
| 3 | Medium IR | On | 0.08 | 3 | 100 | 100 |

In experimental group 1, ten lice and ten eggs were simultaneously exposed to continuous light from a short wave emitter which emitted light over the 0.5 to 5 micron wavelength region, with a peak value at about 1.2 microns. In experimental groups 2 and 3, ten lice and ten eggs were exposed to continuous light from a medium wave emitter which emitted light over the 1 to 7 micron wavelength region, with a peak value at about 2.5 microns. In experimental group 3, an additional band pass filter for the range of 2.8 um to 3.8 um was placed between the emitter and the lice and eggs. Each of the experimental groups included a control group of ten lice and ten eggs. The control lice and eggs were not exposed to light from the emitters, and had between a 0% and a 10% mortality rate in all three groups. It is noted that the level of exposure in experimental groups 2 and 3 is less than or equal to the maximum permissible exposure (MPE) levels generally allowed by health standards.

In an embodiment of the present invention, housing 20 is adapted to function as a handle for system 10. Alternatively, system 10 is adapted to be stand-alone. For some applications, system 10 is incorporated into a comb, a helmet, gloves, or any other lice and eggs removal device. For example, system 10 may comprise gloves, and illumination unit may comprise one or more LEDs. For some applications, optical guide unit 24 comprises light guides that are incorporated into a comb, such as into tines of the comb. Alternatively or additionally, system 10 comprises a hair-raising and/or hair-separation component, to facilitate application of the light to the infestation. For example, the component may comprise a comb, a single-port or multi-port suction applicator, a flexible fastener that can bind to hair (e.g., Velcro), one or more hair clips, or apparatus described in the above-referenced U.S. Pat. No. 5,078,157 to Golan et al., which is incorporated herein by reference.

In an embodiment of the present invention, techniques and apparatus described herein are combined with lice and egg extermination techniques and apparatus described in one or more of the patents and patent application publications described in the Background of the Invention section hereinabove.

Although system 10 has been described herein as appropriate for treating lice infestation, the system may also be used for treating other ectoparasites, such as fleas, ticks, mites, and scabies. Additionally, it is noted that methods and apparatus described herein are generally applicable to ectoparasitic infestations affecting both humans and animals.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treating an infested area of a subject, comprising:
a head cover; and
a plurality of tines coupled to the head cover and comprising respective heating elements that are configured to emit infrared light having a wavelength less than 10 microns.

2. Apparatus for treating an infested area of a subject, comprising:
a housing; and
at least one heating element coupled to the housing and configured to emit light having an emission spectrum selected from the group consisting of: visible light and infrared light, which spectrum is better absorbed by an infestation of the area than by tissue of the subject, the infestation including at least one infestation selected from the group consisting of: lice, and lice eggs, and the tissue including at least one tissue selected from the group consisting of: skin of the subject, and hair of the subject.

3. The apparatus according to claim 2, wherein the infestation includes both lice and eggs, and wherein the heating element is configured to emit the light having the emission spectrum that is better absorbed by the infestation than by the tissue.

4-5. (canceled)

6. The apparatus according to claim 2, wherein the housing comprises a head cover.

7. The apparatus according to claim 6, wherein the housing comprises a plurality of hair separators configured to displace the hair of the subject.

8. The apparatus according to claim 7, wherein the at least one heating element comprises a plurality of heating elements, and wherein at least a portion of the plurality of hair separators comprise the plurality of heating elements.

9-15. (canceled)

16. The apparatus according to claim 7, wherein the plurality of hair separators are spaced apart by a distance of more than 1 mm.

17. The apparatus according to claim 7, further comprising a thermally-conductive material, which is configured to conduct heat from the at least one heating element to the housing.

18-19. (canceled)

20. The apparatus according to claim 17, wherein at least a portion of the plurality of hair separators comprises the thermally-conductive material.

21-24. (canceled)

25. The apparatus according to claim 6, wherein the at least one heating element is configured to raise its temperature to below 70 C.

26. The apparatus according to claim 25, wherein the at least one heating element is configured to raise its temperature to below 60 C.

27. The apparatus according to claim 25, wherein, in response to the raised temperature, the heating element emits light having an emission spectrum that has a peak between 9 microns and 10 microns.

28. The apparatus according to claim 25, wherein the at least one heating element is configured to raise its temperature to between 40 C and 70 C.

29. The apparatus according to claim 28, wherein the at least one heating element is configured to raise its temperature to between 40 C and 55 C.

30. The apparatus according to claim 28, wherein the at least one heating element is configured to raise its temperature to between 55 C and 70 C.

31. (canceled)

32. A method for treating a subject, the method comprising:
identifying an infestation of an area of the subject, the infestation including at least one infestation selected from the group consisting of: lice, and lice eggs; and
emitting light in a vicinity of the infestation by generating heat in the vicinity of the infestation, the light having an emission spectrum selected from the group consisting of: visible light and infrared light, which spectrum is better absorbed by the infestation of the area than by tissue of the subject, the tissue including at least one tissue selected from the group consisting of: skin of the subject, and hair of the subject.

33. The method according to claim 32, wherein generating the heat comprises, responsively to the generating, emitting light having an emission spectrum that has a peak between 9 microns and 10 microns.

34. The method according to claim 32, wherein the infestation includes both lice and eggs, and wherein identifying the infestation comprises identifying the infestation including both lice and eggs.

35-39. (canceled)

40. The method according to claim 32, wherein generating heat in the vicinity of the infestation comprises raising a temperature of at least one heating element to between 40 C and 70 C.

41-44. (canceled)

45. The method according to claim 32, further comprising facilitating application of the emitted light to the infestation by moving the hair of the subject wherein moving the hair comprises separating the hair of the subject.

46-51. (canceled)

52. The method according to claim 32, wherein emitting the light comprises emitting the light from tines.

53-58. (canceled)

59. The apparatus according to claim 1, wherein the plurality of tines are spaced apart by a distance of more than 1 mm.

60. The apparatus according to claim 59, wherein the apparatus is configured to raise a temperature of the plurality of tines to raise its temperature to between 40 C and 70 C.
